(19) United States
(12) Patent Application Publication
(10) Pub. No.: US 2018/0103956 A1
(43) Pub. Date: Apr. 19, 2018

(54) MEDICAL MATERIAL

(71) Applicant: GUNZE LIMITED, Ayabe-shi, Kyoto (JP)

(72) Inventors: Yuki SAKAMOTO, Ayabe-shi, Kyoto (JP); Toshiharu SHINOKA, Tokyo (JP); Hirotsugu KUROBE, Tokushima-shi, Tokushima (JP)

(73) Assignee: GUNZE LIMITED, Ayabe-shi, Kyoto (JP)

(21) Appl. No.: 15/566,500

(22) PCT Filed: Mar. 22, 2016
(86) PCT No.: PCT/JP2016/059790

§ 371 Date: Oct. 13, 2017

(30) Foreign Application Priority Data
Apr. 27, 2015 (JP) 2015-090403

(51) Int. Cl. A61B 17/12; A61L 27/00; A61B 17/00

(52) U.S. Cl. CPC A61B 17/12; A61B 2017/00575; A61B 17/0057; A61L 27/00

(57) ABSTRACT

Provided is a defect hole closing material that has almost no fear of long-term failure and allows minimally invasive treatment for an atrial septal defect. The defect hole closing material is formed of two tubular bodies (a first tubular part and a second tubular par) having a stitch-like structure of a bioabsorbable material, has a sandglass shape, figure-of-eight shape, double spindle shape, or peanut shape, and includes a coil spring of which both ends are respectively engaged with a first end part and a second end part and that is passed through the insides of the first tubular part and the second tubular part from the first end part side to the second end part side via a substantially middle part. When pushing the defect hole closing material out of a catheter, the action of the coil spring causes the first tubular part and the second tubular part to come close with the substantially middle part as a center and expand tube diameters.

### TECHNICAL FIELD

The present invention relates to a medical material for treating a defect hole formed in a biological tissue, and in particular, to a medical material that is set in a catheter, sent to a treatment site through a blood vessel, and placed in an organism.

### BACKGROUND ART

The heart of a human is divided into left and right chambers by a tissue called the septum, in which each of the left and the right chambers has an atrium and a ventricle, and configured to include two atria and two ventricles, i.e., the right atrium, the right ventricle, the left atrium, and the left ventricle. As for the heart having such a configuration, there is a disease called an atrial septal defect (ASD) in which a hole called a defect hole congenitally opens in the atrial septum separating between the right atrium and the left atrium because of a developmental disorder in the fetal stage.

As treatment for the atrial septal defect, the following two methods exist. One is a surgical operation to be performed with the chest cut, and the other one is catheterization using an occluder without cutting the chest.

The surgical operation (a patch operation) uses a cardiopulmonary bypass to open the chest, and closes the defect hole by a patch. The catheterization sets an occluder in a catheter, inserts the catheter into a blood vessel to send the catheter to a target position (the defect hole), and then releases the occluder to place the occluder in the body. The catheterization is one that without incising the chest, from a vein (the femoral vein) at the root of a leg, sends a small jig (device) called the occluder folded in an elongated shape to the position of the hole opening in the atrial septum, and occludes the hole. The advantage of the catheterization is that without performing a thoracotomy requiring general anesthesia, from an inconspicuous point, i.e., the root of a leg (an inguinal region), the treatment can be performed making a tiny skin incision (a few mm).

Japanese Unexamined Patent Publication JP-A2008-512139 (Patent Literature 1) discloses an assembly (an occluder) used for catheterization for an atrial septal defect. This assembly seals a passageway (a defect hole) in the heart. This assembly includes: a closure device for sealing the passageway in the heart including a first anchor used to be placed proximate a first end of the passageway, a second anchor used to be placed proximate a second end of the passageway, and a flexible elongate member adapted to extend through the passageway and used to connect to the first and second anchors, in which the second anchor is capable of movement relative to the elongate flexible member to vary the length of the elongate member between the first and second anchors; and a delivery system for delivering the closure device to the passageway in the heart, in which the delivery system is configured to move within the lumen of a guide catheter and includes a wire configured to control movement of the second anchor along the flexible elongate member.

In addition, Patent Literature 1 discloses that a patent foramen ovale (PFO) closure device (an occluder) includes a left atrial anchor, a right atrial anchor, a tether, and a lock, and the left atrial anchor, the right atrial anchor connected to the left atrial anchor via the tether, and the lock remain in the heart to seal a PFO.

### CITATION LIST

### Patent Literatures

### Patent Literature 1

Japanese Translation of PCT International Application Publication No. 2008-512139

### SUMMARY OF INVENTION

### Technical Problem

In the case of the patch operation, a cardiopulmonary bypass is used, and invasiveness is high, thus causing the problem of a long hospitalization period. In the case of the catheterization, it is preferable because a cardiopulmonary bypass is not used, and invasiveness is low, resulting in a short hospitalization period.

As disclosed in Patent Literature 1, the left atrial anchor and the right atrial anchor remain in the heart. In addition, the left atrial anchor and the right atrial anchor include one or more arms, and the arms extend radially outward from a hub and are preferably formed from a rolled sheet of binary nickel titanium alloy. Further, the left atrial anchor and the right atrial anchor are expanded in an organism to occlude the defect hole; however, once the expansion of the anchors is started, easy restoration is impossible. A dedicated retrieval device having complicated structure and difficult to operate from outside an organism as disclosed in Patent Literature 1 will be used to fold the anchors.

However, in cases such as when an anchor is caught on a biological tissue inside a corresponding atrium to damage the biological tissue, there may be no time to spare for folding the anchor with such a dedicated retrieval device. In such cases, an immediate switch to the thoracotomy must be made. This finally results in the problem of undergoing a highly invasive thoracotomy.

Further, the defect hole occluder made of metal remains in the body through the whole life, and therefore there is the problem of fear of long-term failure.

The present invention is developed in consideration of the above problems, and an object thereof is to provide a medical material that is capable of being released and placed at a treatment site inside an organism, allows minimally invasive catheterization with easy operations without a complicated structure, and has almost no fear of long-term failure even when remaining in the body.

### Solution to Problem

In order to accomplish the above object, the medical material according to the present invention takes the following technical means.

That is, the medical material according to the present invention is a medical material formed of a tubular body having a stitch-like structure using a wire, and the medical material has a shape in which the tube diameter of a substantially middle part of the tubular body is smaller than the tube diameters of the other parts, is formed with a first tubular part on a first end part side in a tubular body longer direction of the medical material and a second tubular part on the other end part side with the substantially middle part as a center, and includes an elastic member of which both ends are respectively engaged with the wire material at the first end part and the wire material at the second end part and that is passed through the insides of the first tubular part and the second tubular part from the first end part side to the second end part side via the substantially middle part.

Preferably, it can be configured that when the elastic member is in a compressed state, the first end part and the second end part come close with the substantially middle part as the center, and the tube diameters of the other parts are expanded.

Further preferably, it can be configured that when the elastic member is in the compressed state, the tube diameters of the other parts are expanded to a size corresponding to a defect hole to be closed by the medical material.

Further preferably, it can be configured that when the elastic member is in a stretched state, the first end part and the second end part separate with the substantially middle part as the center, and the tube diameters of the other parts are reduced.

Further preferably, it can be configured that when the elastic member is in the stretched state, the tube diameters of the other parts are reduced to a size corresponding to a catheter in which the medical material is to be contained.

Further preferably, it can be configured that the elastic member is a coil spring having a diameter smaller than the tube diameter of the substantially middle part.

Further preferably, it can be configured that an end part of the elastic member is joined to a small tubular part that is provided outside of the tubular body having the stitch-like structure and screwable with an operation wire.

Further preferably, it can be configured that the shape is a sandglass shape, a figure-of-eight shape, or a double spindle shape.

Further preferably, it can be configured that the wire is of a bioabsorbable material.

Further preferably, it can be configured that a porous tubular layer formed of any of a non-woven fabric, a sponge, and a film made of a bioabsorbable material, and a composite body of them is arranged on the inner surface of the tubular body.

### Advantageous Effects of Invention

The medical material of the present invention is capable of being released and placed at a treatment site inside an organism and allows minimally invasive catheterization with easy operations without a complicated structure. In addition, the medical material of the present invention has little fear of long-term failure even when remaining in the body.

### BRIEF DESCRIPTION OF DRAWINGS

FIG. 1 is an overall view of a defect hole closing material 100 as an example of the medical material according to the present invention (a coil spring 140 is in a compressed state).

FIG. 2 is an overall view of the defect hole closing material 100 as the example of the medical material according to the present invention (the coil spring 140 is in an intermediate state).

FIG. 3 is an overall view of the defect hole closing material 100 as the example of the medical material according to the present invention (the coil spring 140 is in a stretched state).

FIG. 4 is an overall view of the defect hole closing material 100 as the example of the medical material according to the present invention (the coil spring 140 is in the compressed state and in the stretched state).

FIG. 5A is a partial side view of the defect hole closing material 100 in FIG. 2.

FIG. 5B is a cross-sectional view along A-A in FIG. 5A.

FIG. 6 is a conceptual view of a case where the defect hole closing material 100 as the example of the medical material according to the present invention is used for catheterization for an atrial septal defect.

FIG. 7 is an enlarged view (part 1) of a part B in FIG. 6 showing the procedure of the catheterization.

FIG. 8 is an enlarged view (part 2) of the part B in FIG. 6 showing the procedure of the catheterization.

FIG. 9 is an enlarged view (part 3) of the part B in FIG. 6 showing the procedure of the catheterization.

FIG. 10 is an overall view of a defect hole closing material 400 as an example of a medical material according to a variation of the present invention (the coil spring 140 is in the compressed state).

FIG. 11 is an overall view of the defect hole closing material 400 as the example of the medical material according to the variation of the present invention (the coil spring 140 is in the intermediate state).

FIG. 12 is a partial enlarged view of FIG. 11.

FIG. 13 is a drawing substitute photograph for explaining the case where the defect hole closing material 400 as the example of the medical material according to the variation of the present invention is applied to an animal experiment.

### DESCRIPTION OF EMBODIMENTS

In the following, the medical material according to the present invention will be described in detail on the basis of the drawings. Note that in the following, as an example of the medical material according to the present invention, a defect hole closing material used for catheterization is described, but also suitable for closing another opening or passage, for example, another opening in the heart, such as a ventricular septal defect or patent ductus arteriosus, or an opening or passage at another site in an organism (e.g., the stomach), such as arteriovenous fistula. Accordingly, a defect hole closing material according to an embodiment of the present invention is not limited to being used for closing an atrial septal defect hole.

Further, in the following embodiment, the stitch-like structure of the defect hole closing material (an occluder) 100 is described as an object obtained by knitting a bioabsorbable fiber (an example of a wire); however, the present invention is not limited to this. It is only necessary to be a defect hole closing material allowing catheterization adapted to close a defect hole formed in an organism, and its stitch-like structure may be knitted with a wire other than the bioabsorbable fiber as long as the wire is of a material characterized by the below-described first to third characteristics and exhibits the first action to the third action. In order to keep form retainability (shape retainability), such a wire preferably has a certain level of hardness.

### [Configuration]

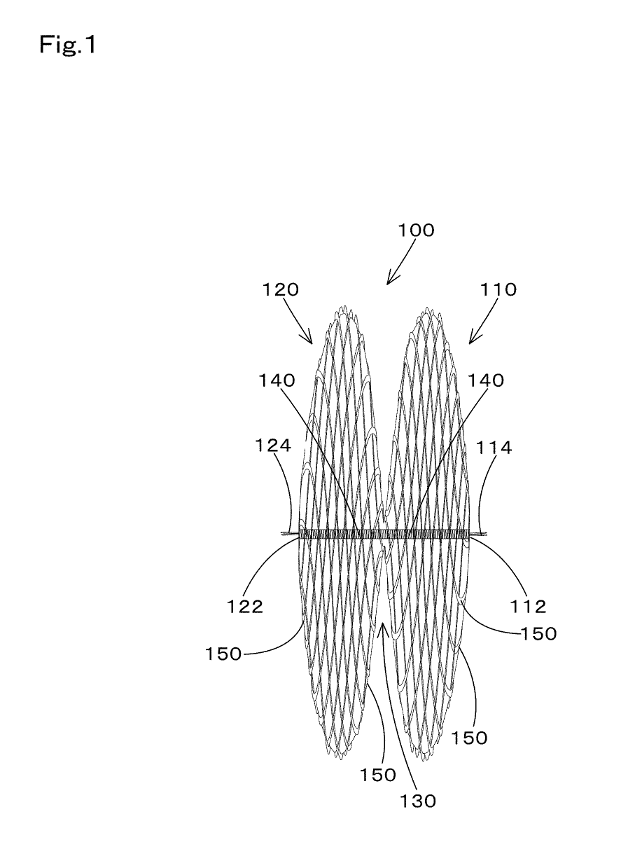

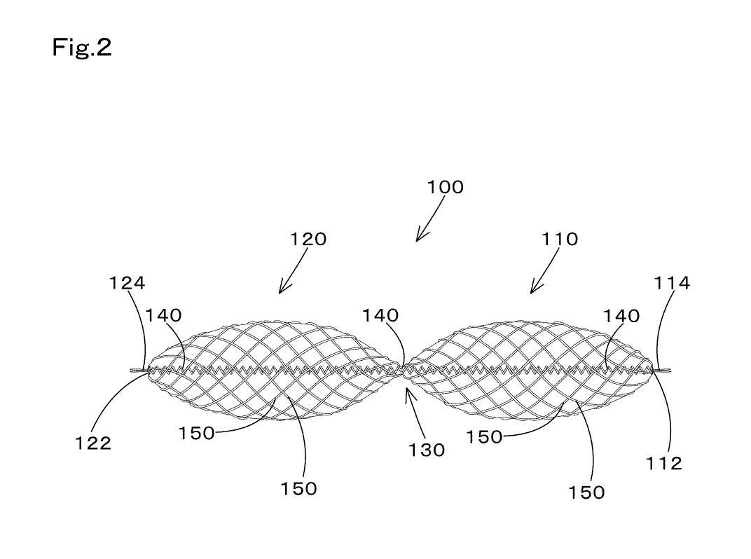

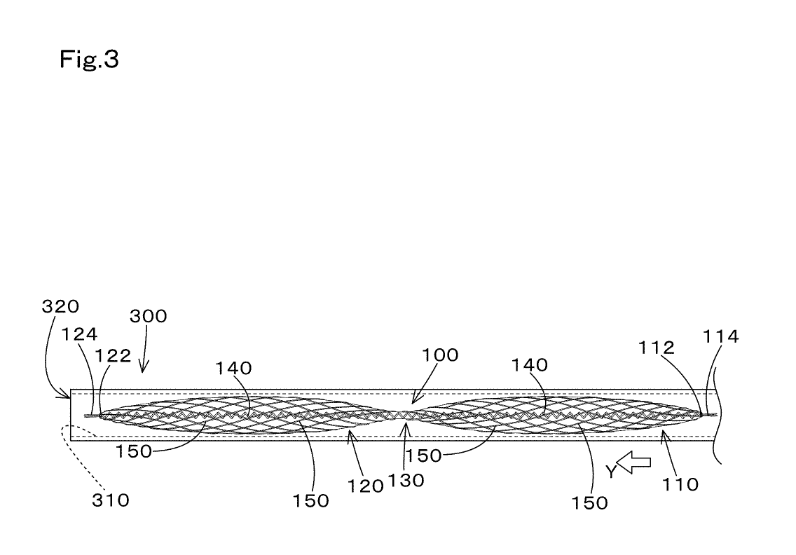

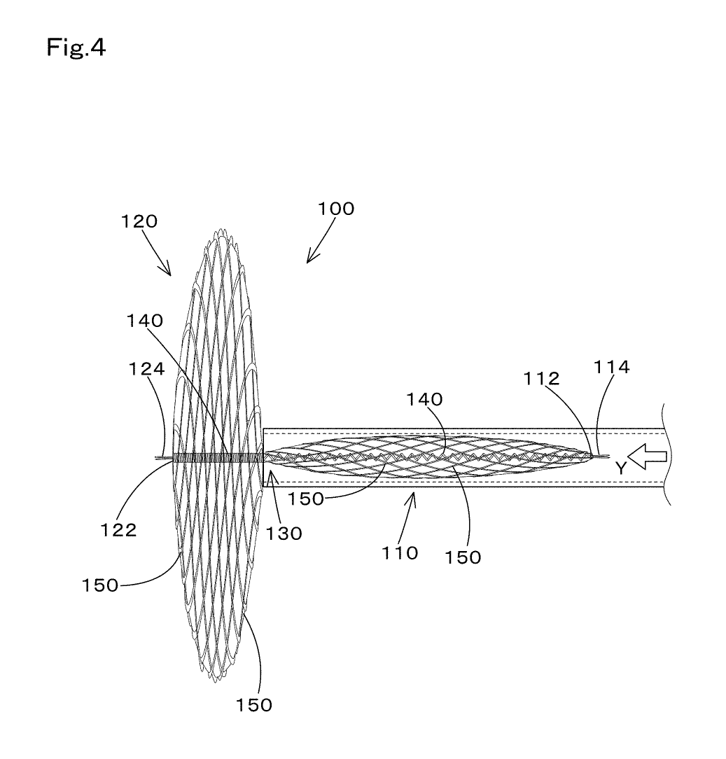

FIG. 1 illustrates an overall view of the defect hole closing material 100 according to the present invention (a coil spring 140 is in a compressed state), FIG. 2 illustrates an overall view of the defect hole closing material 100 (the coil spring 140 is in an intermediate state), FIG. 3 illustrates an overall view of the defect hole closing material 100 (the coil spring 140 is in a stretched state), and FIG. 4 illustrates an overall view of the defect hole closing material 100 (the coil spring 140 is in the compressed state and in the stretched state). In addition, FIG. 3 is a diagram illustrating a state where the whole of the defect hole closing material 100 is contained in a catheter 300, and FIG. 4 is a diagram illustrating a state where half of the defect hole closing material 100 (a first tubular part 110 side) is contained in the catheter 300. Pushing the defect hole closing material 100 wholly contained inside (space formed by the inner wall 310) of the catheter 300 illustrated in FIG. 3 from the first tubular 110 side in a Y direction indicated by an arrow to push out a second tubular part 120 from an opening part 320 of the catheter 300 results in the state of FIG. 4, and further pushing out the first tubular part 110 in the Y direction indicated by the arrow results in the state of FIG. 1. Note that the state of the defect hole closing material 100 illustrated in FIG. 2 is the intermediate state of the coil spring 140 between the compressed state and the stretched state and a virtual state.

As illustrated in these views, the defect hole closing material 100 is roughly formed of a tubular body having a stitch-like structure using the wire, has a shape in which the tube diameter of a substantially middle part 130 of the tubular body is smaller than the tube diameters of the other parts, and is formed with: the first tubular part 110 on a first end part 112 side in the tubular body longer direction of the defect hole closing material 100; and the second tubular body 120 on the other end part (a second end part 122) side with the substantially middle part 130 as a center. In addition, a characteristic point is to include the coil spring 140 as an example of an elastic member of which both ends are respectively engaged with a wire 114 at the first end part 112 and with a wire 124 at a second end part 122 and that is passed through the insides of the first tubular part 110 and the second tubular part 120 from the first end part 112 side to the second end part 122 side via the substantially middle part 130. Even in the case other than the coil spring 140, the elastic member is only required to be a member that has elasticity and can exhibit the below-described actions on the basis of the elasticity, but is not limited to the coil spring 140.

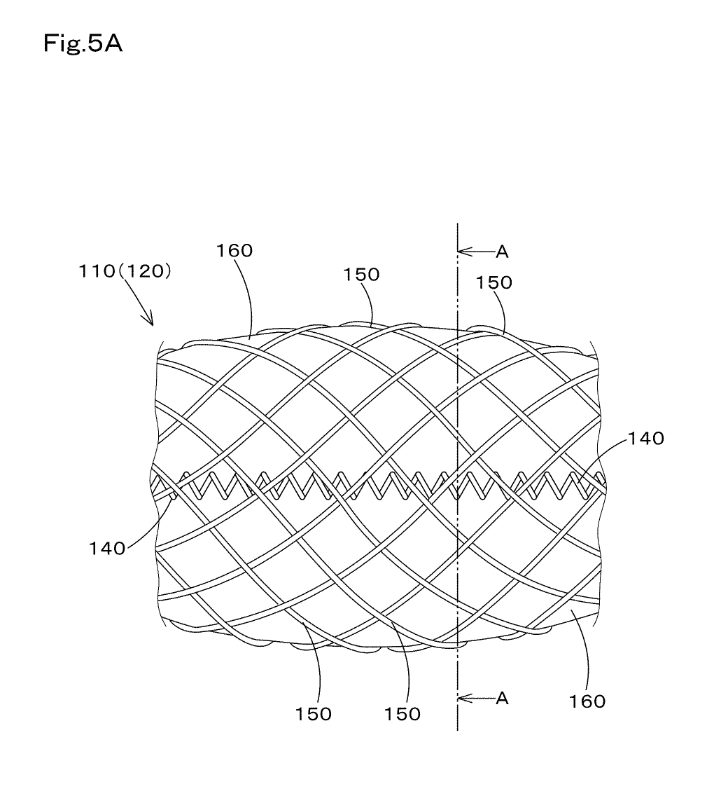

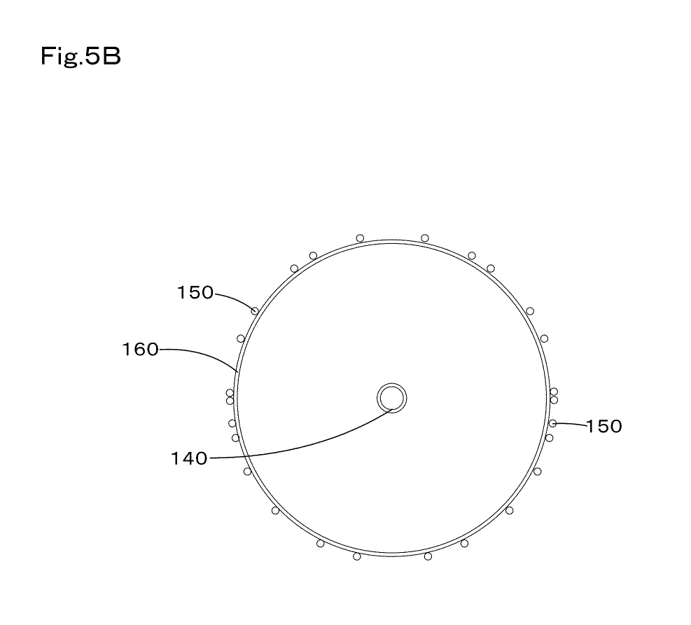

Further, FIG. 5A illustrates a partial side view of the defect hole closing material 100 and FIG. 5B illustrates a cross-sectional view along A-A in FIG. 5A. Note that although FIG. 5B is the cross-sectional view of the defect hole closing material 100, FIG. 5B illustrates only the cross-sections of the coil spring 140, a bioabsorbable fiber 150, and a porous tubular layer 160 but does not illustrate the stitches of the bioabsorbable fiber 150 viewable from an A direction indicated by an arrow. In addition, in FIG. 1 to FIG. 5, in order to facilitate the understanding of the presence of the coil spring 140 and the stitches of the bioabsorbable fiber 150, the porous tubular layer 160 is illustrated as a transparent material.

As illustrated in these views (in particular, in FIG. 2), the defect hole closing material 100 is formed of the two tubular bodies (the first tubular part 110 and the second tubular part 120) having the stitch-like structure using a bioabsorbable material, and the shape thereof is a shape formed of such two tubular bodies and called, for example, a sandglass shape, a figure-of-eight shape, a double spindle shape (the shape of two continuous long rod-like spindle-shaped objects whose middles are thick and both ends are narrow), or a peanut shape (the external shape of a peanut shell containing two nuts). The defect hole closing material 100 having such a shape has a shape in which the substantially middle part 130 is narrowed so as to make the tube diameter of the substantially middle part 130 smaller than the tube diameters of the other parts. That is, the first tubular part 110 on the first end part 112 side and the second tubular part 120 on the second end part 122 side are formed with the substantially middle part 130 as the center.

In addition, although not limited to, the first tubular part 110 and the second tubular part 120 are integrally knitted such that the tube diameter of the substantially middle part 130 is made smaller than the tube diameters of the other parts, and the defect hole closing material 100 is formed in the sandglass shape, figure-of-eight shape, double spindle shape, or peanut shape formed of the two tubular bodies as the whole shape of the defect hole closing material 100. In this case, the whole shape of the defect hole closing material 100 is formed by using a frame (a three-dimensional paper pattern) of such a sandglass shape, figure-of-eight shape, double spindle shape, or peanut shape to knit the one bioabsorbable fiber 150 in conformity with the frame. Further, although not limited to, the defect hole closing material 100 may be formed in the sandglass shape, figure-of-eight shape, double spindle shape, or peanut shape formed of the two tubular bodies as the whole shape of the defect hole closing material 100 by integrally knitting the first tubular part 110 and the second tubular part 120 to knit a tubular body having a substantially uniform diameter, then binding and/or heat-setting the substantially middle part 130 to thereby form the shape in which the tube diameter of the substantially middle part 130 is smaller than the tube diameters of the other parts, and then releasing the binding and/or releasing the heat-setting of the substantially middle part 130 to form the substantially middle part 130 of which the tube diameter is larger than the diameter of the coil spring 140. Still further, although described in detail below, forming such a shape makes it possible to cause the change in shape, i.e., when pushing the defect hole closing material 100 wholly contained inside (the space formed by the inner wall 310) of the catheter 300 illustrated in FIG. 3 from the first tubular part 110 side to push out the second tubular part 120 from the opening part 320 of the catheter 300 in the Y direction indicated by the arrow, the second tubular part 120 is released from the space formed by the inner wall 310 of the catheter 300 to compress the coil spring 140 in the second tubular part 120, thus resulting in the state of FIG. 4, and when further pressing out the first tubular part 110 in the Y direction indicated by the arrow, the first tubular part 110 is released from the space formed by the inner wall 310 of the catheter 300 to compress the coil spring 140 in the first tubular part 110, thus resulting in the state of FIG. 1.

In addition, the defect hole closing material 100 is such that the one end thereof is engaged with the first end part 112 (e.g., caught on a loop of the wire 114 at the first end part 112), the other end part is engaged with the second end part 122 (e.g., caught on a loop of the wire 124 at the second end part 122), and the coil spring 140 passed through the insides of the first tubular part 110 and the second tubular part 120 from the first end part 112 side to the second end part 122 side via the substantially middle part 130 is provided. In addition, the looped wire 114 and wire 124 are formed of the bioabsorbable fiber 150.

As illustrated in FIG. 1, when the coil spring 140 is in the compressed state, the first end part 112 and the second end part come close with the substantially middle part 130 as the center, and the tube diameters of the first tubular part 110 and the second tubular part 120 as the other parts other than the substantially middle part 130 are expanded. Particularly preferably, when the coil spring 140 is in the compressed state, the tube diameters of the first tubular part 110 and the second tubular part 120 as the other parts other than the substantially middle part 130 are expanded to a size corresponding to a defect hole to be closed by the defect hole closing material 100.

In addition, as illustrated in FIG. 3, when the coil spring 140 is brought into the stretched state by containing the defect hole closing material 100 in the catheter 300, or the like, the first end part 112 and the second end part 122 separate with the substantially middle part 130 as the center, and the tube diameters of the first tubular part 110 and the second tubular part 120 as the other parts are reduced. Particularly preferably, when the coil spring 140 is in the stretched state, the tube diameters of the first tubular part 110 and the second tubular part 120 as the other parts are reduced to a size corresponding to the catheter 300 in which the defect hole closing material 100 is to be contained.

As described, by using the coil spring 140 having the diameter smaller than the tube diameter of the substantially middle part 130, the first end part 112 and the second end part 122 as the other end part in the longer direction of the tubular bodies in the defect hole closing material 100 can be brought close or separated. When bringing the coil spring 140 into the compressed state, as illustrated in FIG. 1, the first end part 112 and the second end part 122 come close to expand the tube diameters of the parts other than the substantially middle part 130 (the tube diameters of body parts of the first tubular part 110 and the second tubular part 120), and when bringing the coil spring 140 into the stretched state, as illustrated in FIG. 3, the first end part 112 and the second end part 122 separate to reduce the tube diameters of the parts other than the substantially middle part 130 (the tube diameters of the body parts of the first tubular part 110 and the second tubular part 120). Further, as illustrated in FIG. 4, when pushing the second tubular part 120 out of the catheter 300 in the Y direction indicated by the arrow, the second tubular part 120 of which the shape was restricted by the inner wall 310 of the catheter 300 can freely change the shape, and only the part of the coil spring 140 contained in the second tubular part 120 is compressed to expand only the tube diameter of the body part of the second tubular part 120. Still further, when pushing the first tubular part 110 out of the catheter 300 in the Y direction indicated by the arrow, the first tubular part 110 of which the shape was restricted by the inner wall 310 of the catheter 300 can also freely change the shape, and the part of the coil spring 140 contained in the first tubular part 110 is also compressed to, as illustrated in FIG. 1, expand the tube diameter of the body part of the first tubular part 110 as well.

Note that in the defect hole closing material 100, the porous tubular layer 160 formed of any of a non-woven fabric, sponge, and film made of the bioabsorbable material, and a composite body of them is arranged on the inner surfaces of the tubular bodies. The first tubular part 110 and the second tubular part 120 are formed of a woven fabric (open one), knitted fabric, braid-like fabric, or tubular knitted fabric of the bioabsorbable fiber 150, and wholly formed as the stitch-like structure. Here, to describe for confirmation, the stitch-like structure includes, without limitation to a knitted fabric formed by knitting, a mesh-like structure formed as an open woven structure like a screen door as described above. That is, the structure called “stitch-like” and the structure called “mesh-like” are both allowable. The porous tubular layer 160 is formed of any of a non-woven fabric, a sponge, a film, and a composite body of them in order to hold a drug by application, impregnation, embedding, or the like. Further, the porous tubular layer 160 is not limited to being of the bioabsorbable material but may be of a non-bioabsorbable material.

As described, basically except for the coil spring 140, the first tubular part 110, the second tubular part 120, and the porous tubular layer 160 are all formed of the bioabsorbable material, and therefore the whole of the defect hole closing material 100 except for the coil spring 140 has bioabsorbability. In addition, the change in the shape of the defect hole closing material 100 allows the treatment to close a defect hole to be performed, and therefore the defect hole closing material 100 is formed using a material, stitch shape, fiber structure, and fiver cross section that even when the shape of the defect hole closing material 100 is changed in an organism as described, prevent an in vivo tissue from being damaged.

Note that the coil spring 140 typically uses, for example, a nickel-titanium alloy or the like, and does not have bioabsorbability, but may use the below-described magnesium-based alloy to have bioabsorbability. Using an alloy having bioabsorbability for the coil spring 140 is advantageous in terms of reacting to X-ray imaging, and using an alloy not having bioabsorbability is advantageous in terms of preventing the problem of fear of long-term failure because no metallic member remains in the body through the whole life.

The bioabsorbable fiber 150 forming the first tubular part 110 and the second tubular part 120 is of at least one type selected from a polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), glycolic acid-lactide (D, L, and DL isomers)-ε-caprolactone copolymers, or the like, and used processed into the form of any of a monofilament yarn, multifilament yarn, twisted yarn, braid, and the like, preferably used in the form of the monofilament yarn.

Further, the material of the bioabsorbable fiber 150 may be a biodegradable alloy. As an example of such a biodegradable alloy, an alloy based on magnesium as a raw material can be cited.

The diameter of the bioabsorbable fiber 150 is approximately 0.001 mm to 1.5 mm, and a fiber diameter and type suitable for catheterization to be applied are selected. Also, the cross section of the bioabsorbable fiber 150 may be any of a circle, an ellipse, and other different shapes (such as a star shape) on condition that the damage to an in vivo tissue is prevented. Further, the surface of the bioabsorbable fiber 150 may be subjected to hydrophillic treatment by plasma discharge, electron beam treatment, corona discharge, ultraviolet irradiation, ozone treatment, or the like. Still further, the bioabsorbable fiber 150 may be subjected to application or impregnation treatment with an X-ray non-permeable material (such as barium sulfide, a gold ship, or a platinum chip), attachment treatment with a drug (such as a drug suitable for catheterization for an atrial septal defect), or coating treatment with a natural polymer such as collagen or gelatin, or a synthetic polymer such as polyvinyl alcohol or polyethylene glycol.

The first tubular part 110 and the second tubular part 120 are such that the bioabsorbable fiber 150 is, for example, fabricated into braid-like woven fabrics using a braiding machine having multiple (e.g., 8 or 12) feeders around a silicone-made rubber tube (illustration is omitted) having an outside diameter desired as a monofilament yarn, or knitted into the tubular body having the substantially uniform diameter and the stitch-like structure using a circular knitting machine (illustration is omitted). After the knitting, as described above, the narrowing is performed in the substantially middle part 130 with a strap made of the same material as the first tubular part 110 and the second tubular part 120, thus forming the sandglass shape, figure-of-eight shape, double spindle shape, or peanut shape formed of the two tubular bodies. The tube diameters of the first tubular part 110 and the second tubular part 120 are smaller than the inside diameter of the catheter when reduced, and when expanded, have a size suitable for the catheterization for an atrial septal defect. For example, when expanded, the tube diameters of the first tubular part 110 and the second tubular part 120 are 5 mm to 80 mm, preferably approximately 15 mm to 25 mm. In addition, the lengths of the first tubular part 110 and the second tubular part 120, and the density of the stitch-like structure of the defect hole closing material 100 are also density suitable for the catheterization for an atrial septal defect. Note that the tube diameters and lengths of the first tubular part 110 and the second tubular part 120 do not have to be the same, but may be changed so as to be suitable for the catheterization for an atrial septal defect.

The bioabsorbable material forming the porous tubular layer 160 is not particularly limited, and synthetic absorbable polymers can be cited, such as a polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), and glycolic acid-lactide (D, L, and DL isomers)-ε-caprolactone copolymers. These may be used individually, or two or more types may be used together. Among them, at least one type selected from the group consisting of a polyglycolic acid, lactide (D, L, and DL isomers)-ε-caprolactone copolymers, a glycolic acid-ε-caprolactone copolymer, and glycolic acid-lactide (D, L, and DL isomers)-ε-caprolactone copolymers is suitable due to exhibiting appropriate degradation behavior, and the porous tubular layer 160 is formed of any of a non-woven fabric, sponge, film, and composite body of them. In particular, as a preferred embodiment, the non-woven fabric can be exemplified.

Further, the material of the porous tubular layer 160 may be a biodegradable alloy. As an example of such a biodegradable alloy, an alloy based on magnesium as a raw material can be cited.

When the porous tubular layer 160 is of a non-woven fabric, hydrophilic treatment may be applied. The hydrophilic treatment is not particularly limited, and for example, plasma treatment, glow discharge treatment, corona discharge treatment, ozone treatment, surface grafting treatment, ultraviolet irradiation treatment, and the like can be cited. Among them, the plasma treatment is suitable because the plasma treatment can dramatically improve a water absorption rate without changing the appearance of the non-woven fabric layer. Note that the porous tubular layer 160 may be a sponge layer, film layer, composite layer of a non-woven fabric and a sponge layer, composite layer of a non-woven fabric and a film layer, composite layer of a sponge layer and a film layer, or composite layer of a non-woven fabric, a sponge layer, and a film layer.

The porous tubular layer 160 holds a drug suitable for the catheterization for an atrial septal defect.

As described above, the defect hole closing material 100 according to the present invention has the following characteristics.

(First characteristic) Being formed in the sandglass shape, figure-of-eight shape, double spindle shape, or peanut shape formed of the first tubular part 110 and the second tubular part 120 separated by the narrowing in the substantially middle part 130.
(Second characteristic) The one end is engaged with the first end part 112 (caught on the looped wire 114 at the first end part 112), whereas the other end is engaged with the second end part 122 (caught on the looped wire 124 at the second end 122), and the coil spring 140 passed through the insides of the first tubular part 110 and the second tubular part 120 from the first end part 112 side to the second end part 122 side via the substantially middle part 130 is provided.
(Third characteristic) Being formed of the first tubular part 110, the second tubular part 120, the coil spring 140 (when formed of a magnesium-based alloy), and the porous tubular layer 160, and these components are all formed of the bioabsorbable material (the coil spring 140 does not necessarily have to have bioabsorbability).

In addition, the first characteristic and the second characteristic allows: on the defect hole closing material 100 contained in the catheter 300, when pushing the second tubular part 120 out of the catheter 300, the second tubular part 120 of which the shape was restricted by the inner wall 310 of the catheter 300 to freely change the shape, and only the part of the whole of the coil spring 140 contained in the second tubular part 120 to be compressed to expand only the tube diameter of the body part of the second tubular part 120, and when further pushing the first tubular part 110 out of the catheter 300, the first tubular part 110 of which the shape was restricted by the inner wall 310 of the catheter 300 to also freely change the shape, and the part of the whole of the coil spring 140 contained in the first tubular part 110 to be also compressed to expand the tube diameter of the body part of the first tubular part 110 as well.

In particular, the defect hole closing material 100 is suitable for the catheterization for an atrial septal defect in terms of exhibiting the following actions.

(First action) Being able to be set in the catheter 300 by stretching the whole of the coil spring 140 to thereby make the tube diameter of the defect hole closing material 100 smaller than the inside diameter of the catheter 300.
(Second action) Being able to be set in the catheter 300 and sent to a position of a hole opening in the atrial septum, when pushing the first end part 112 with an applicator or the like in an organism to push the second tubular part 120 from the catheter 300 into the organism, compress the coil spring 140 in the second tubular part 120 to expand the tube diameter of the body part of the second tubular part 120, and when further pushing the first end part 112 with the applicator or the like to push the first tubular part 110 from the catheter 300 into the organism, also compress the coil spring 140 in the first tubular part 110 to expand the tube diameter of the body part of the first tubular part 110 as well, and bring the first tubular part 110 arranged on the right atrium side and the second tubular part 120 arranged on the left atrium side close with the substantially middle part 130 as the center to occlude the hole opening in the atrial septum.
(Third action) Since the material forming the defect hole closing material 100 (the coil spring 140 may be excluded) is wholly the bioabsorbable material, and finally absorbed in the organism, fear of long-term failure is almost eliminated.

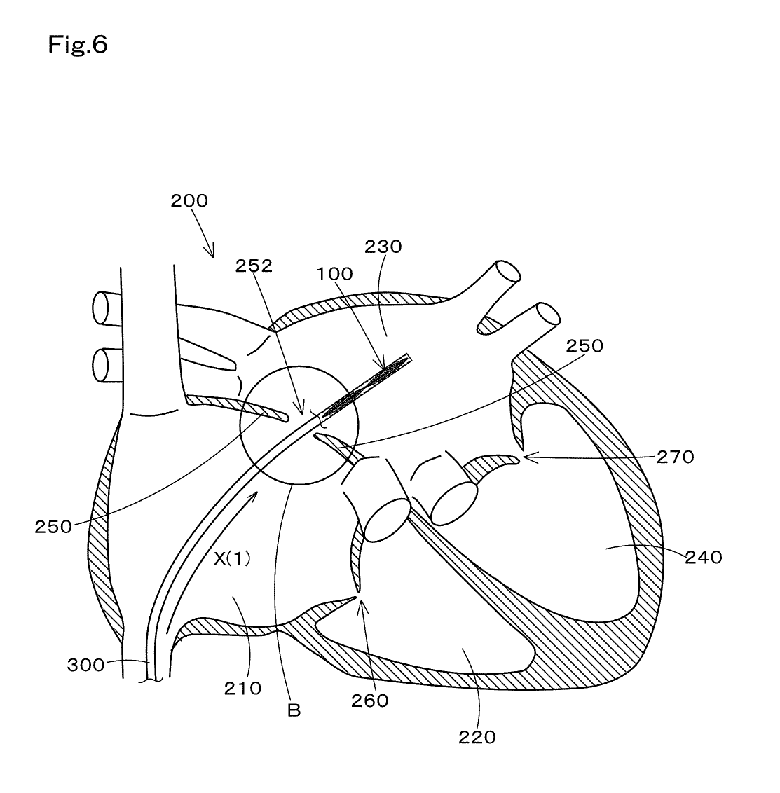

For easily understanding such actions, the case where the defect hole closing material 100 is used for the catheterization for an atrial septal defect will be described with reference to FIG. 6 to FIG. 9.

### [Usage Mode]

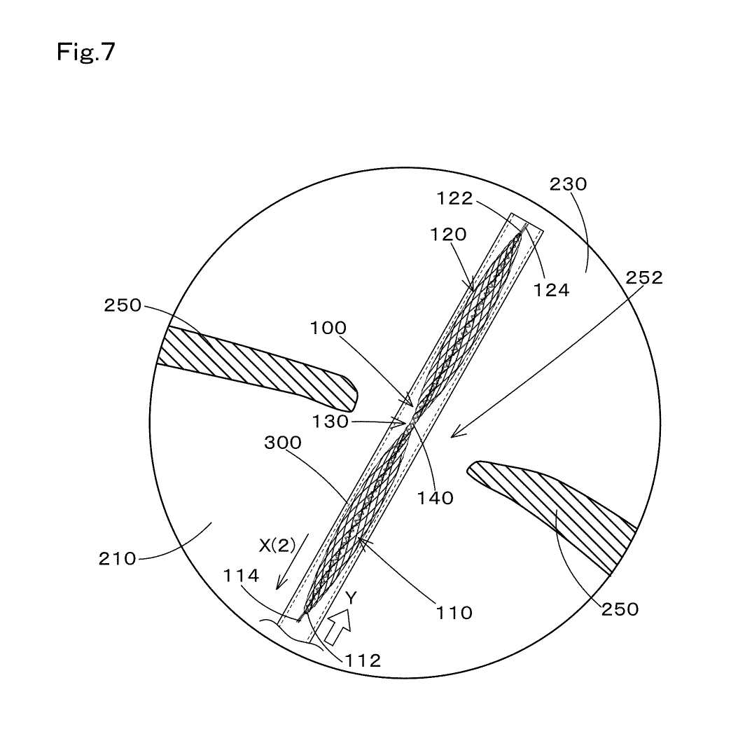

FIG. 6 illustrates a conceptual view of a case where the defect hole closing material 100 is used for the catheterization for the atrial septal defect, and FIG. 7 to FIG. 9 illustrate enlarged views of a part B in FIG. 6 showing the procedure of the catheterization. Note that in the following, only matters specific to the usage mode of the defect hole closing material 100 according to the present embodiment are described, and since the description of general matters is the same as that of publicly-known catheterization for an atrial septal defect, detailed description is not repeated here.

As illustrated in FIG. 6, the heart 200 of a human is configured to include two atria and two ventricles, i.e., the right atrium 210 connected to the superior vena cava and the inferior vena cava to receive venous blood from the whole body, the right ventricle 220 connected to the right atrium 210 via the pulmonary artery and the tricuspid valve 260 to send venous blood to the lungs, the left atrium 230 connected to the pulmonary vein to receive arterial blood from the lungs, and the left ventricle 240 connected to the left atrium 230 via the aorta and the mitral valve 270 to send arterial blood to the whole body. The atrial septal defect is a disease in which a defect hole 252 opens in the atrial septum 250 separating between the right atrium 210 and the left atrium 230. Note that in FIG. 6, to facilitate understanding, the tip side of the catheter 300 is indicated by a virtual line and the defect hole closing material 100 contained in the catheter 300 is indicated by a solid line.

First, outside an organism, a pull is exerted in a direction to separate the first end part 112 and the second end part 122 of the defect hole closing material 100 capable of being expanded to a size appropriate for the defect hole 252, and thereby the whole of the coil spring 140 is stretched to make the tube diameter of the defect hole closing material 100 smaller than the inside diameter of the catheter 300 for setting in the catheter 300. The catheter 300 containing the defect hole closing material 100 is inserted through the femoral vein (see FIG. 3), the catheter 300 is moved in an X(1) direction indicated by an arrow and passed through the defect hole 252 from the right atrium 210 side, and the catheter 300 containing the defect hole closing material 100 is brought close to the left atrium 230 side.

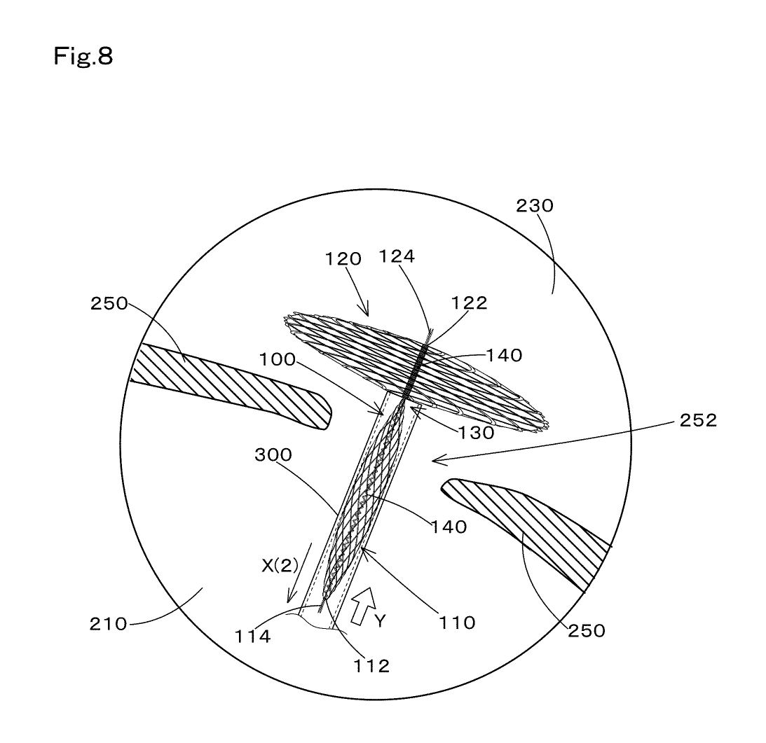

As illustrated in FIG. 6 and FIG. 7, at a position where the substantially middle part 130 of the defect hole closing material 100 faces the vicinity of the defect hole 252, the catheter 300 containing the defect hole closing material 100 is stopped. When inside the organism, pushing the second tubular part 120 out of the catheter 300 with an applicator or the like in a Y direction indicated by an arrow, the second tubular part 120 of which the shape was restricted by the inner wall 310 of the catheter 300 can freely change the shape, and only the part of the coil spring 140 contained in the second tubular part 120 is compressed to expand only the tube diameter of the body part of the second tubular part 120 as illustrated in FIG. 8.

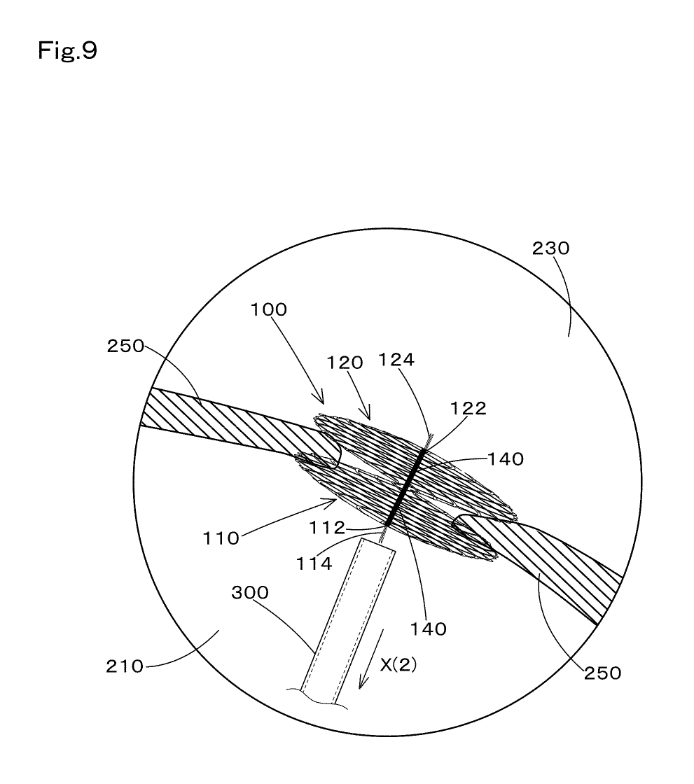

In addition, when further pushing the first tubular part 110 out of the catheter 300 with the applicator or the like in the Y direction indicated by the arrow, the first tubular part 110 of which the shape was restricted by the inner wall 310 of the catheter 300 can also freely change the shape, and the part of the coil spring 140 contained in the first tubular part 110 is also compressed to, as illustrated in FIG. 9, expand the tube diameter of the body part of the first tubular part 110 as well.

That is, when pushing the defect hole closing material 100 out of the catheter 300 with the applicator or the like, the second tubular part 120 arranged on the left atrium side is first expanded, and then the first tubular part 110 arranged on the right atrium side is expanded. As a result, the first tubular part 110 arranged on the right atrium side and the second tubular part 120 arranged on the left atrium side come close with the substantially middle part 130 (the defect hole 252) as the center, and also the tube diameter of the first tubular part 110 and the tube diameter of the second tubular part 120 are expanded. Finally, as illustrated in FIG. 9, the first tubular part 110 and the second tubular part 120 sandwich the atrial septum 250 from both side of the atrial septum 250, and the defect hole 252 opening in the atrial septum 250 can be occluded by the defect hole closing material 100.

After that, the catheter 300 is moved in an X(2) direction indicated by an arrow, and the catheter 300 is taken out of the organism to complete the treatment. In doing so, inside the organism (to be accurate, near the defect hole 252), the defect hole closing material 100 wholly formed of the bioabsorbable material (the coil spring 140 may be excluded) is placed. As described, since the material of the defect hole closing material 100 placed in the organism is wholly the bioabsorbable material (the coil spring 140 may be excluded) and finally absorbed in the organism, there is almost no fear of long-term failure.

In addition, when the coil spring 140 is not provided, before placing the defect hole closing material 100 in the organism, it is necessary to fix the form of the defect hole closing material 100 to the form illustrated in FIG. 9, and it has been considered that for example, the bioabsorbable fiber 150 is adapted to have thermal adhesiveness to heat set the bioabsorbable fiber 150 in the organism. However, the present defect hole closing material 100 is advantageous because the coil spring 140 enables the form of the defect hole closing material 100 to be fixed to the form illustrated in FIG. 9.

Since the defect hole closing material 100 according to the present embodiment is wholly formed of the bioabsorbable material (the coil spring 140 may be excluded), and finally absorbed in an organism as described above, there is almost no fear of long-term failure. Also, since providing the coil spring 140 allows the tube diameter of the defect hole closing material 100 to easily change, the setting in the catheter becomes possible by changing the tube diameter of the defect hole closing material 100 to be smaller. Further, only by pushing the defect hole closing material 100 out of the catheter 300 at the position of a defect hole, the change can be easily made so as to increase the tube diameter of the defect hole closing material 100 and bring the two tubular bodies close because of the presence of the coil spring 140, and the resulting form can be easily fixed to occlude the defect hole opening in the atrial septum.

### <Variation>

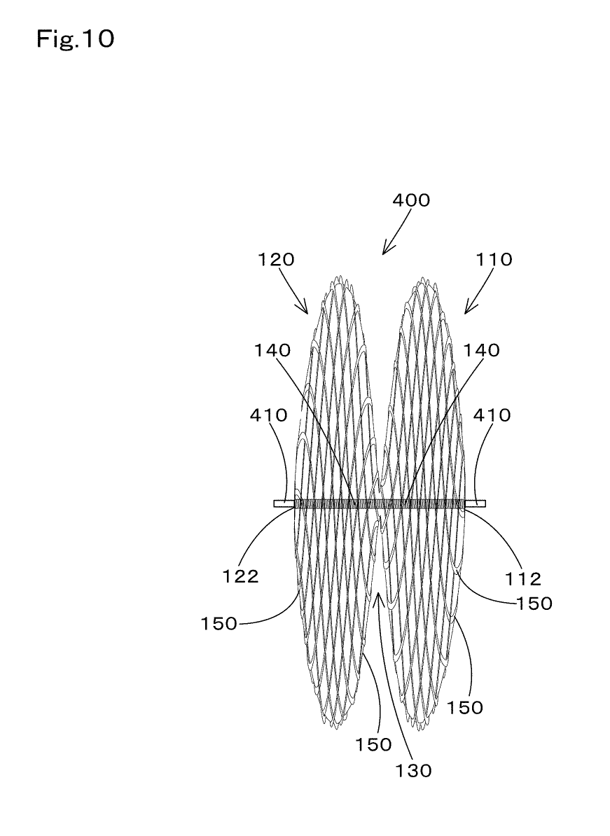

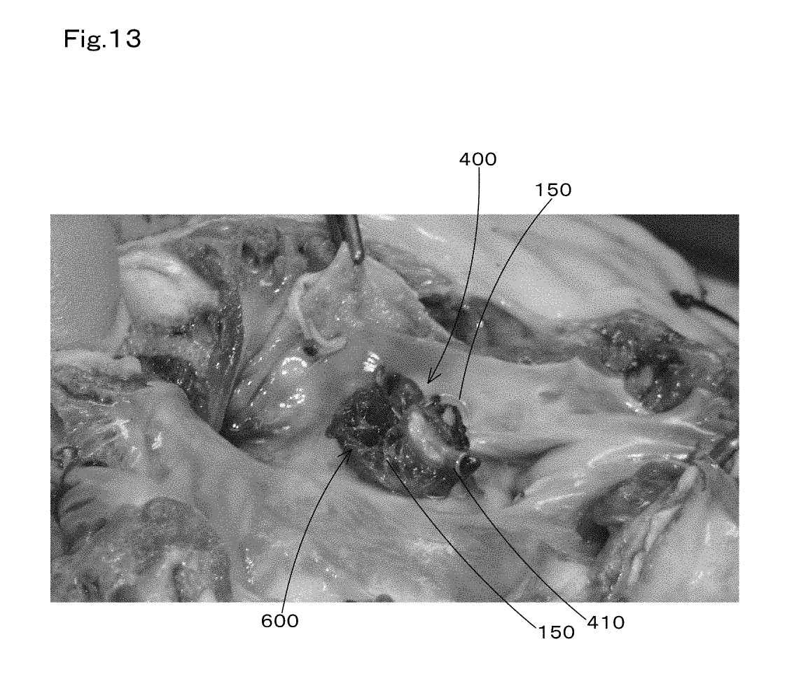

In the following, a defect hole closing material (an occluder) 400 as an example of a medical material according to a variation of the present invention will be described with reference to FIG. 10 to FIG. 13. Note that the defect hole closing material 400 according to the present variation is the same as the above-described defect hole closing material 100 except that the end parts of the elastic member (the coil spring 140) of the above-described defect hole closing material 100 are joined to small tubular provided on the outer sides of the tubular bodies (the first tubular part 110 and the second tubular part 120) having the stitch-like structure and screwable with an operation wire 500, and therefore parts overlapping with the above-described description are not repeated here.

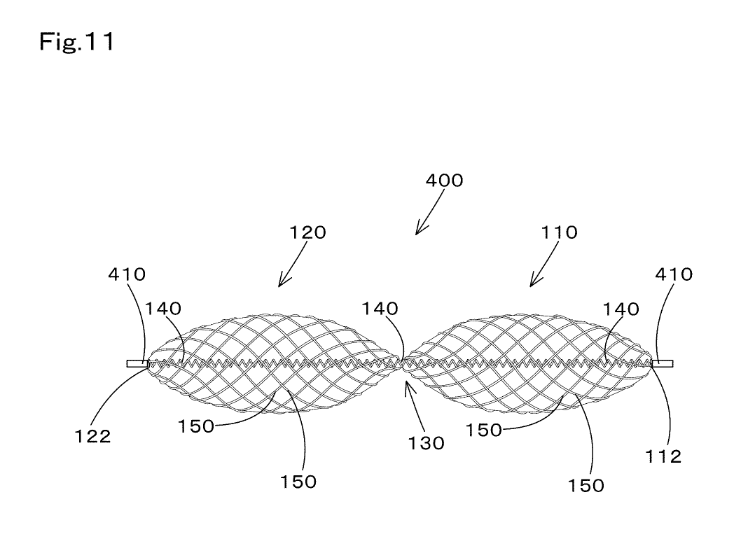

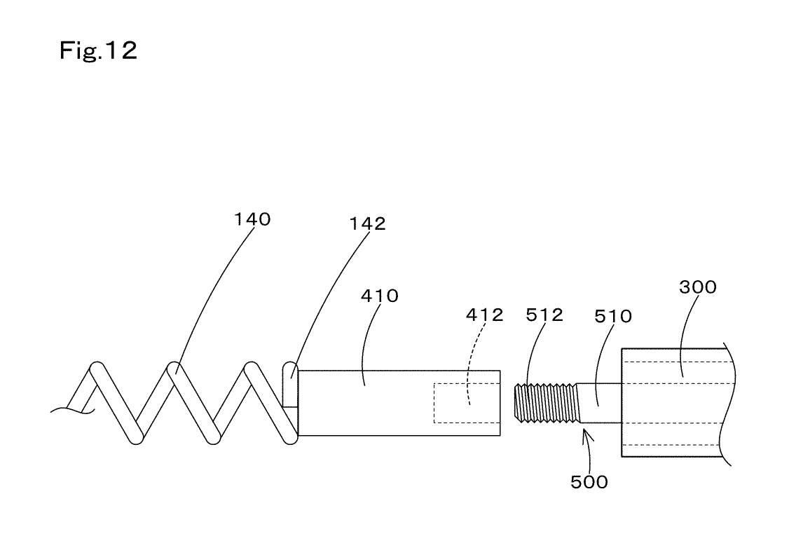

FIG. 10 illustrates a diagram that is an overall view of the defect hole closing material 400 (the coil spring 140 is in the compressed state) and corresponds to FIG. 1, FIG. 11 illustrates a diagram that is an overall view of the defect hole closing material 400 (the coil spring 140 is in the intermediate state) and corresponds to FIG. 2, and FIG. 12 illustrates a partial enlarged view of FIG. 11.

As illustrated in these views, both end parts 142 of the coil spring 140 are joined to the small tubular parts (more specifically, tubular metal pieces 410) having female screw parts 412 screwable with a male screw part 512 provided at the tip part 510 of the operation wire 500 to be inserted inside the catheter 300. The metal pieces 410 are provided on the outer sides of the tubular bodies (the first tubular part 110 and the second tubular part 120) having the stitch-like structure. As in the defect hole closing material 100, the coil spring 140 is such that the one end of the coil spring 140 is engaged with the first end part 112 (e.g., caught on the loop of the wire 114 at the first end part 112) and the other end is engaged with the second end part 122 (e.g., caught on the loop of the wire 124 at the second end part 122). In addition, the coil spring 140 of which both end parts 142 are joined with the metal pieces 410 is passed through the insides of the first tubular part 110 and the second tubular part 120 from the first end part 112 side to the second end part 122 side via the substantially middle part 130. Note that small tubular parts may be of a material other than metal, a metal piece 410 may be joined not to both of the end parts 142 of the coil spring 140 but to only one end, and the metal pieces 410 may have male screw parts, whereas the operation wire 500 may have a female screw part.

Further, it can be exemplified that the coil spring 140 employs a nickel-titanium alloy as described above and the metal pieces 410 employ stainless steel. As a joining method for the combination of such metals, caulking joining can be exemplified.

The defect hole closing material 400 having the structure as described above is used in the same manner as the usage mode of the above-described defect hole closing material 100. Particularly preferably, since in the defect hole closing material 400, both end parts 142 of the coil spring 140 and the metal pieces 410 having the female screw parts 412 screwable with the male screw part 512 provided at the tip part 510 of the operation wire 500 to be inserted inside the catheter 300 are joined, the following usage is possible.

As illustrated in FIG. 7, outside an organism, a pull is exerted in a direction to separate the first end part 112 and the second end part 122 of the defect hole closing material 100 capable of being expanded to a size appropriate for the defect hole 252, and thereby the whole of the coil spring 140 is stretched to make the tube diameter of the defect hole closing material 100 smaller than the inside diameter of the catheter 300 for setting in the catheter 300. At this time, although not illustrated in FIG. 7, the male screw part 512 provided at the tip part 510 of the operation wire 500 inserted inside the catheter 300 and a female screw part 412 of one of the metal pieces 410 joined to both end parts 142 of the coil spring 140 are screwed together.

Then, inside the organism, as illustrated in FIG. 7 or FIG. 8, the second tubular part 120 or the first tubular part 110 is pushed out of the catheter 300 in the Y direction indicated by the arrow. In this case, since the coil spring 140 is connected to the tip of the operation wire 500 via the metal piece 410 (since the bioabsorbable fiber 150 is connected with the inflexible metallic member), the pushing out in the Y direction indicated by the arrow can be performed with good operability by operating the operation wire 500 from outside the organism.

Then, as illustrated in FIG. 9, the tube diameter of the body part of the second tubular part 120 and the tube diameter of the body part of the first tubular part 110 of the defect hole closing material 400 are both expanded. After that, although not illustrated in FIG. 9, the operation wire 500 is operated (rotated) from outside the organism to release the screwing between the male screw part 512 and the female screw part 412. Subsequently, the catheter 300 and the operation wire 500 are moved in the X(2) direction indicated by the arrow, and the catheter 300 and the operation wire 500 are taken out of the organism to complete the treatment.

Specific effects of performing the treatment in this manner will be described with reference to FIG. 12. FIG. 12 is a diagram for explaining the case where the defect hole closing material 400 is applied to an animal experiment, and a drawing substitute photograph showing a state of an affected area two months after the treatment was performed using the defect hole closing material 400 on an animal (sheep) simulating a human having an atrial septal defect of the heart.

As illustrated in FIG. 12, it turns out that around the bioabsorbable fiber 150 of the defect hole closing material 400, a biological tissue 600 is formed to occlude the defect of the atrial septum. Further, in this case, it was able to be confirmed that in the right atrium and in the left atrium, none of a thrombus, mitral regurgitation (MR), and tricuspid regurgitation (TR) was developed.

The defect hole closing material 400 according to the variation can further improve the operability of the above-described defect hole closing material 100 in the above manner.

Note that the embodiment disclosed this time should be considered to be an exemplification in all respects, but not to be a limited one. The scope of the present invention is shown not by the above description but by claims, and it is intended that meanings equivalent to claims and all modifications within the scope are included.

### INDUSTRIAL APPLICABILITY

The present invention is suitable for a medical material to be set in a catheter to treat a defect hole formed in a biological tissue, and particularly preferable in terms of being capable of being released and placed at a treatment site to perform minimally invasive treatment, and having almost no fear of long-term failure even when remaining in the body.

### REFERENCE SIGNS LIST

100, 400 Medical material (occluder)
110 First tubular part
112 First end part
120 Second tubular part
122 Second end part
130 Substantially middle part
140 Coil spring
150 Bioabsorbable fiber
160 Porous tubular layer
200 Heart
250 Atrial septum
252 Defect hole
300 Catheter
500 Operation wire

1. A medical material formed of a tubular body having a stitch-like structure using a wire,
the medical material
having a shape in which a tube diameter of a substantially middle part of the tubular body is smaller than tube diameters of other parts,
being formed with a first tubular part on a first end part side in a tubular body longer direction of the medical material and a second tubular part on the other end part side with the substantially middle part as a center, and
comprising an elastic member of which both ends are respectively engaged with the wire material at the first end part and the wire material at the second end part and that is passed through insides of the first tubular part and the second tubular part from the first end part side to the second end part side via the substantially middle part.

2. The medical material according to claim 1, wherein
when the elastic member is in a compressed state, the first end part and the second end part come close with the substantially middle part as the center, and the tube diameters of the other parts are expanded.

3. The medical material according to claim 2, wherein
when the elastic member is in the compressed state, the tube diameters of the other parts are expanded to a size corresponding to a defect hole to be closed by the medical material.

4. The medical material according to claim 1, wherein
when the elastic member is in a stretched state, the first end part and the second end part separate with the substantially middle part as the center, and the tube diameters of the other parts are reduced.

5. The medical material according to claim 4, wherein
when the elastic member is in the stretched state, the tube diameters of the other parts are reduced to a size corresponding to a catheter in which the medical material is to be contained.

6. The medical material according to claim 1, wherein
the elastic member is a coil spring having a diameter smaller than the tube diameter of the substantially middle part.

7. The medical material according to claim 1, wherein
an end part of the elastic member is joined to a small tubular part that is provided outside of the tubular body having the stitch-like structure and screwable with an operation wire.

8. The medical material according to claim 1, wherein
the shape is a sandglass shape, a figure-of-eight shape, or a double spindle shape.

9. The medical material according to claim 1, wherein
the wire is of a bioabsorbable material.

10. The medical material according to claim 1, wherein
a porous tubular layer formed of any of a non-woven fabric, a sponge, and a film made of a bioabsorbable material, and a composite body of them is arranged on an inner surface of the tubular body.
